(19) United States
(12) Patent Application Publication
(10) Pub. No.: US 2012/0130217 A1
(43) Pub. Date: May 24, 2012

(54) MEDICAL DEVICES HAVING ELECTRODES MOUNTED THEREON AND METHODS OF MANUFACTURING THEREFOR

(76) Inventors: James V. Kauphusman, Champlin, MN (US); Martin M. Grasse, Boston, MA (US); Allan M. Fuentes, Mound, MN (US); Salome A. Gonzalez, Crystal, MN (US)

(21) Appl. No.: 12/952,948

(22) Filed: Nov. 23, 2010

(51) Int. Cl. A61B 5/04; H01R 43/16; A61N 1/05

(52) U.S. Cl. 600/373; 607/119; 29/874

(57) ABSTRACT

Medical devices, methods of manufacturing medical devices, and systems comprising medical devices are provided. The device comprises a shaft having a major lumen sized to receive a second medical device and an electrode mounted thereon. The shaft includes an inner liner and outer layer. The method of manufacture includes forming a shaft by forming an inner liner and an outer layer by covering the inner liner with a polymeric material. The method further includes mounting an electrode onto the shaft of the device, and then heating and cooling the shaft. The system comprises a medical device having a shaft and an electrode mounted thereon. The shaft has a major lumen sized to receive another device. The system further comprises an electronic control unit configured to receive signals from the electrode and to determine a position of the electrode and/or monitor electrophysiological data.

### BACKGROUND OF THE INVENTION

a. Field of the Invention

This disclosure relates to a family of medical devices. More particularly, this disclosure relates to medical devices, such as, for example, deflectable catheter-introducers or sheaths, having one or more electrodes mounted thereon for electrophysiology (EP) diagnostics and localization and visualization of said devices, as well as methods of manufacturing and systems with which such medical devices are used, including robotic surgical systems.

b. Background Art

It is well known to use a medical device called a sheath or catheter-introducer when performing various therapeutic and/or diagnostic medical procedures on or in the heart, for example. Once inserted into a patient's body, these particular medical devices (hereinafter referred to as “sheaths”) provide a path through a patient's vasculature to a desired anatomical structure or site for a second medical device, such as, for example, a catheter, a needle, a dilator, etc., and also allow for the proper positioning or placement of the second medical device relative to the desired anatomical structure.

One drawback to conventional sheaths and their use is that visualization of the sheath and/or its position has proved difficult, if not impossible. As a result, physicians have been unable to see the sheath and/or its position during the performance of a medical procedure without the use of ionizing radiation (e.g., acute x-ray delivery via a fluoroscope). However, with the advent and growing use of various automated guidance systems, such as, for example, magnetic-based and robotic-based guidance systems, the need for such visualization capability has increased. More particularly, it is important for the physician/clinician operating such automated systems to know and understand exactly where the various medical devices being used are located and how they are oriented.

In addition to the need of visualization in the use of automated guidance systems, the need for this capability is also increasing in instances where a physician manually controls medical devices. For example, for procedures performed on the left side of the heart, a transseptal puncture is used to cross the septum separating the right atrium from the left atrium. In such procedures, a long, small diameter needle is passed down a lumen in the sheath and is used to puncture the septal wall. Once formed, the sheath is inserted into the hole created by the puncture operation and crosses through the septum, thereby providing another medical device within the sheath access to the left atrium. Using current visualization systems, such as, for example, fluoroscopy, the transseptal crossing point (and the sheath therein) is invisible to the physician. Accordingly, if the physician loses visual contact with a device or the transseptal access is interrupted due to, for example, patient movement or the manipulation of a medical device used with the sheath, regaining access increases the procedure time and also may require another puncture of the septum. Because there is no visualization of the sheath, or any representation of the sheath on a display the physician is using, the physician has no reference to help guide him to regain access.

Accordingly, the inventors herein have recognized a need for sheath designs and methods of manufacturing that minimize and/or eliminate one or more of the deficiencies in conventional cardiac catheter-introducers and sheaths.

### BRIEF SUMMARY OF THE INVENTION

The present invention is directed to a family of medical devices, such as deflectable cardiac catheter-introducers and sheaths. These medical devices typically comprise a shaft having a proximal end, a distal end, and a major lumen disposed therein extending between the proximal and distal ends and configured to receive a second medical device therethrough. The medical device further comprises at least one electrode mounted on the shaft thereof.

In an exemplary embodiment, the shaft of the medical device is formed of a number of constituent parts. The shaft includes an inner liner having an inner surface and an outer surface, wherein the inner surface of the inner liner forms or defines the major lumen of the shaft. The shaft further includes an outer layer adjacent to the outer surface of the inner liner. In an exemplary embodiment, the outer layer has at least one minor lumen coupled thereto in which one or more electrical wires of the electrode(s) mounted on the shaft are disposed. The minor lumen in the outer layer extends from the proximal end of the shaft to a location on the shaft near where the electrode is mounted. In an exemplary embodiment, the outer layer further has one or more additional minor lumens coupled thereto and offset from the at least one minor lumen within which one or more electrical wires are disposed. Deflection elements such as, for example, pullwires, are disposed within these additional and offset lumens.

In accordance with another aspect of the invention, a method of manufacturing a medical device is provided. The method, in accordance with present teachings, includes forming a shaft of the medical device by forming an inner liner having a tubular shape and an inner and outer surface, and forming an outer layer by covering the inner liner with a polymeric material. The method further includes mounting an electrode onto the shaft of the medical device. The method still further includes heating the shaft to a temperature at which the polymeric material melts, and then cooling the shaft.

In accordance with yet another aspect of the invention, a system for performing at least one of a therapeutic and a diagnostic medical procedure is provided. In accordance with this disclosure the system comprises a first medical device having an elongate shaft and at least one electrode mounted on the shaft. The shaft of the medical device comprises a proximal end, a distal end, and a major lumen therein extending between the proximal and distal ends of the shaft. The major lumen is sized and configured to receive a second medical device, such as, for exemplary purposes only, an electrophysiological catheter, a needle, a dilator, and the like.

The system further comprises an electronic control unit (ECU). The ECU is configured to receive signals from the electrode mounted on the shaft of the medical device and, in response to those signals, to automatically determine a position of the electrode and/or monitor electrophysiological data.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a perspective view of an exemplary embodiment of a medical device in accordance with present teachings.

FIGS. 2 and 3 are cross section views of the medical device illustrated in FIG. 1 taken along the lines 2/3-2/3 showing the shaft of the medical device in various stages of assembly.

FIG. 4 is side view of a portion of an exemplary embodiment of the medical device illustrated in FIG. 1.

FIG. 5 is a cut-away perspective view of a portion of the medical device illustrated in FIG. 1.

FIG. 6 is a diagrammatic and schematic view of another exemplary embodiment of the medical device illustrated in FIG. 1 showing the medical device used in connection with an exemplary embodiment of an automated guidance system.

FIG. 7 is a diagrammatic and schematic view of the medical device illustrated in FIG. 5, wherein the distal end of the medical device is deflected.

FIG. 8 is a flow diagram illustrating an exemplary embodiment of a method of manufacturing a medical device in accordance with present teachings.

FIG. 9 is a diagrammatic view of a system for performing at least one of a diagnostic and a therapeutic medical procedure in accordance with present teachings.

FIG. 10 is a simplified diagrammatic and schematic view of the visualization, navigation, and/or mapping system of the system illustrated in FIG. 9.

FIG. 11 is an exemplary embodiment of a display device of the system illustrated in FIG. 8 with a graphical user interface (GUI) displayed thereon.

### DETAILED DESCRIPTION OF EMBODIMENTS OF THE INVENTION

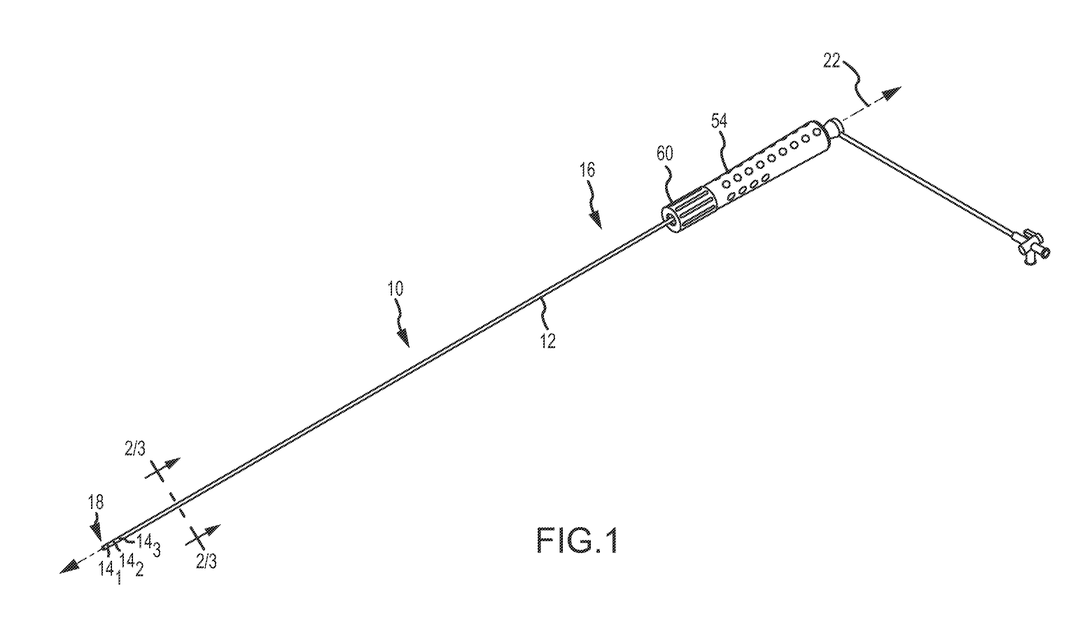

Referring now to the drawings wherein like reference numerals are used to identify identical components in the various views, FIG. 1 illustrates one exemplary embodiment of a medical device 10, such as, for example and without limitation, a sheath or cather-introducer for use in connection with a number of diagnostic and therapeutic procedures performed, for example, within the heart of a human being or an animal. For clarity and brevity purposes, the description below will be directed solely to a medical device 10 that comprises a sheath (sheath 10) for use in cardiac applications. It will be appreciated by those having ordinary skill in the art, however, that the description below may be applicable to medical devices other than sheaths, and for sheaths and medical devices used in connection with applications other than cardiac applications. Accordingly, medical devices other than sheaths, and medical devices/sheaths for use in applications other than cardiac applications, remain within the spirit and scope of the present invention.

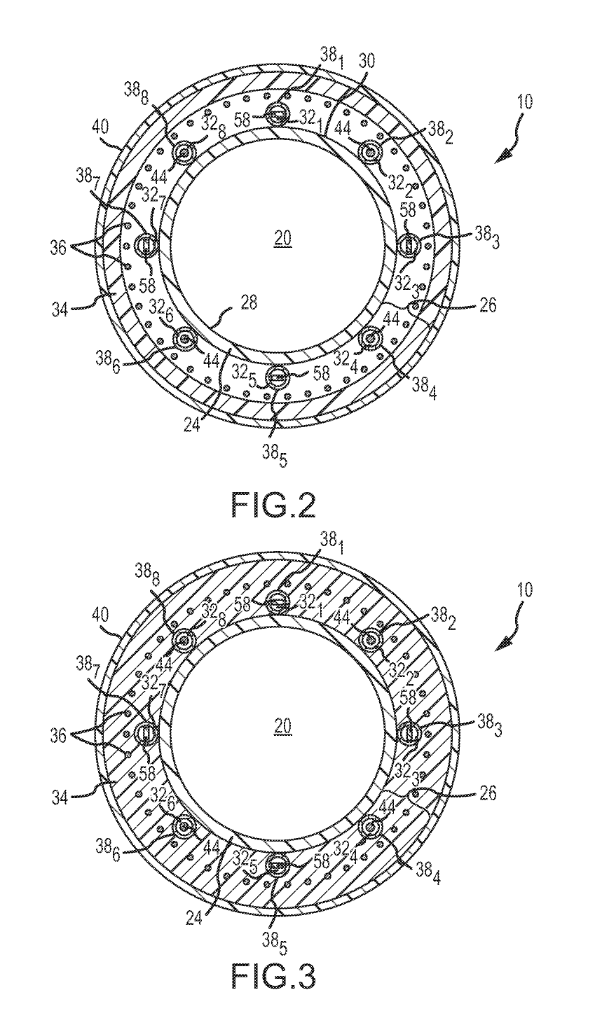

With reference to FIG. 1, in an exemplary embodiment, the sheath 10 comprises an elongate tubular shaft 12 and one or more electrodes 14 (e.g., 14₁, 14₂, 14₃ in FIG. 1) mounted thereon. The shaft 12 has a proximal end 16, a distal end 18, and a major lumen 20 (best shown in FIGS. 2 and 3) extending between proximal and distal ends 16, 18 (as used herein, “proximal” refers to a direction toward the end of the sheath 10 near the physician/clinician, and “distal” refers to a direction away from the physician/clinician). The major lumen 20 defines a longitudinal axis 22 of the sheath 10, and is sized to receive a medical device therein. As illustrated in FIG. 1, and as will be described in greater detail below, the electrodes 14 are mounted on the shaft 12 at the distal end 18 thereof. However, in another exemplary embodiment, one or more of the electrodes 14 may be mounted at a location on the shaft 12 more proximal than the distal end 18. Additionally, the shaft 12 may have straight configuration, or alternatively, may have a fixed curve shape/configuration. The shaft 12 is configured for insertion into a blood vessel or another anatomic structure.

FIGS. 2 and 3 are cross-section views of an exemplary embodiment of the shaft 12, wherein FIG. 2 illustrates the shaft 12 at a non-final stage of assembly, and FIG. 3 illustrates the shaft 12 at a final stage of assembly following the performance of a reflow process on at least a portion of the shaft 12. In this embodiment, and in its most general form, the shaft 12 comprises an inner liner 24 and an outer layer 26.

The inner liner 24 has an inner surface 28 and an outer surface 30, wherein the inner surface 28 defines the major lumen 20. In an exemplary embodiment, the inner liner 24 is formed of extruded polytretrafluoroethylene (PTFE) tubing, such as, for example, Teflon® tubing. In one exemplary embodiment, the PTFE comprises etched PTFE. An inner liner formed of this particular material creates a lubricious lumen (lumen 20) within which other medical devices used with the sheath 10, such as, for example, catheters, needles, dilators, and the like, can be passed. The inner liner 24 is relatively thin. For example, in one embodiment, the inner liner 24 has a thickness on the order 0.0015 inches (0.0381 mm). It will be appreciated by those having ordinary skill in the art that the inner liner 24 may be formed of a material other than PTFE, or etched PTFE. For example, in other exemplary embodiments, the inner layer 24 is comprised of polymeric materials, such as, for example and without limitation, polyether block amides, nylon, and other thermoplastic elastomers. Accordingly, sheaths having inner liners made of materials other than PTFE remain within the spirit and scope of the present disclosure.

With continued reference to FIGS. 2 and 3, the outer layer 26 is disposed adjacent to the inner layer 24, and the outer surface 30 thereof, in particular. In an exemplary embodiment, the outer layer 26 includes one or more minor lumens 32 (i.e., lumens 32₁-32₈ in FIGS. 2 and 3) therein and coupled thereto adapted to receive and house, as will be described in greater detail below, deflectable elements, such as, for example, steering or pull wires associated with a steering mechanism for the sheath 10, or elongate conductors (e.g., electrical wires) coupled to the electrodes 14. Because the major lumen 20 of the shaft 12 must be kept open to allow for the uninhibited passage of other medical devices therethrough, the minor lumens 32 are disposed within the outer layer 26 of the shaft 12.

The outer layer 26 may be formed of a single polymeric material, or alternatively, a combination of different components/materials (e.g., various tubing and braid assemblies) that, after the application of a reflow process on at least a portion of the shaft 12, combine to form the outer layer 26. In the exemplary embodiment illustrated in FIG. 2, the outer layer 26 comprises one or more layers of polymeric material that are placed over the inner liner 24. The polymeric material may be in the form of one or more extruded polymer tube(s) 34 sized so as to fit over the inner layer 24. The polymer tube 34 may comprise one or more of any number of polymeric materials, such as, for example and without limitation, polyether block amides (e.g., Pebax®), polyamides (e.g., nylon), PTFE, etched PTFE, and other thermoplastic elastomers.

The polymer tube 34 may be formed of a single piece of tubing or multiple pieces of tubing. Whether formed of a single piece or multiple pieces, the tube 34 may have a uniform hardness or durometer throughout. Alternatively, different portions of the tube 34 may have different durometers (e.g., the shaft 12 may have a variable durometer from the proximal end 16 to the distal end 18). In an embodiment wherein the tube 34 is formed of multiple pieces, the pieces may be affixed together end to end, or portions of adjacent pieces may overlap each other. These pieces may be coupled or bonded together to form the shaft 12 during a reflow process performed thereon. Additionally, in an exemplary embodiment, one or more portions of the tube 34 disposed at the distal end 18 of the shaft 12, or at any other location on the shaft 12 at or near where an electrode 14 is mounted, are formed so as to be translucent or transparent. The use of transparent or translucent material allows one to locate and access the minor lumen(s) 32 in the outer layer 26 for purposes that will be described in greater detail below.

In an exemplary embodiment, and as illustrated in FIGS. 2 and 3, the outer layer 26 further comprises a braided wire assembly 36 disposed adjacent to and between both the inner liner 24 and the polymeric material or tube 34. The arrangement and configuration of the braided wire assembly 36 and the tube 34 is such that the polymeric material of the tube 34 melts and flows into the braid of the braided wire assembly 36 during a reflow process performed on the shaft 12. The braided wire assembly 36, which may extend the entire length of the shaft 12 (i.e., from the proximal end 16 to the distal end 18) or less than the entire length of the shaft 12, maintains the structural integrity of the shaft 12, and also provides an internal member to transfer torque from the proximal end 16 to the distal end 18 of the shaft 12.

In an exemplary embodiment, the braided wire assembly 36 comprises a stainless steel braid wherein each wire of the braid has a rectangular cross-section with the dimensions of 0.002 inches×0.006 inches (0.051 mm×0.152 mm). It will be appreciated by those having ordinary skill in the art, however, that the braided wire assembly 36 may be formed of material other than, or in addition to, stainless steel. For example, in another exemplary embodiment, the braided wire assembly 36 comprises a nickel titanium (also known as nitinol) braid. Additionally, the braided wire assembly 36 may have dimensions or wire sizes and cross-sectional shapes other than those specifically provided above, such as, for example, a round or circular cross-sectional shape, and also include varying braid densities throughout. Different braid wire sizes allow different shaft torque and mechanical characteristics. Accordingly, braided wire assemblies comprising materials other than stainless steel, and/or dimensions other than those set forth above, remain within the spirit and scope of the present disclosure.

As briefly described above, in an exemplary embodiment, the outer layer 26 further includes one or more minor lumens 32 disposed therein and coupled thereto. Each minor lumen 32 is adapted to receive and house either an electrical wire(s) associated with an electrode 14, or a deflectable element, such as a pull wire, of the steering mechanism of the sheath 10. In an exemplary embodiment, the sheath 10 includes one or more extruded tubes 38 (i.e., 38₁-38₈ in FIGS. 2 and 3), each one of which defines a corresponding minor lumen 32. The tubes 38, which are also known as spaghetti tubes, may be formed of a number of materials known in the art, such as, for example and without limitation, PTFE. In an exemplary embodiment, the tubes 38 are formed a material having a melting point higher than that of the material in polymer tube 34 so that the tubes 38 will not melt when the shaft 12 is subjected to a reflow process. In the embodiment illustrated in FIG. 2, the tubes 38 are affixed or bonded to the outer surface 30 of the inner layer 24. The tubes 38 may be affixed in a number of ways, such as, for example, using an adhesive. One suitable adhesive is cyanoacrylate. As illustrated in FIG. 3, once the shaft 12 is subjected to a reflow process, the polymeric material of the tube 34 surrounds and encapsulates the tubes 38 resulting in the tubes 38, and therefore the minor lumens 32, being disposed within the outer layer 26.

The minor lumens 32 extend axially relative to the longitudinal axis 22 of the sheath 10. In an exemplary embodiment, some or all of the minor lumens 32 that house electrical wires associated with the electrodes 14 (i.e., lumens 32₂, 32₄, 32₆, 32₈ in FIGS. 2 and 3) extend from the proximal end 16 of the shaft 12 to the distal end 18. In another exemplary embodiment, some or all of the minor lumens 32 extend from the proximal end 16 of the shaft 12 to various points or locations on the shaft 12 between the proximal and distal ends 16, 18. For example and with reference to FIG. 1, the minor lumen 32 that houses the electrical wire of the electrode 14₃ may extend from the proximal end 16 of the shaft 12 to the distal end 18. Alternatively, it may extend from the proximal end 16 to the point on the shaft 12 at or near where the electrode 14₃ is mounted. Similarly, minor lumens 32 that house the pull wires of the steering mechanism of the sheath 10 (i.e., the lumens 32₁, 32₃, 32₅, 32₇ in FIGS. 2 and 3) may extend from the proximal end 16 of the shaft 12 to the distal end 18. Alternatively, they may extend from the proximal end 16 to a point in the shaft 12 that the pull wire is coupled to another component of the steering mechanism.

In addition to the above, in an exemplary embodiment, the shaft 12 of the sheath 10 may further include a layer 40 of heat shrink material on the outer surface thereof. With continued reference to FIGS. 2 and 3, the heat shrink material layer 40 is disposed adjacent to the polymeric material of the outer layer 26 (e.g., the polymer tube 34) such that the outer layer 26 is disposed between the inner liner 24 and the heat shrink material layer 40. The heat shrink material layer 40 may be formed of a number of different types of heat shrink materials. In an exemplary embodiment, the heat shrink material layer 40 comprises a fluoropolymer or polyolefin material, and more particularly, a tube formed of such a material sized to fit over the outer layer 26 of the shaft 16. One example of a suitable material for the heat shrink layer 40 is fluorinated ethylene propylene (FEP).

As will be described in greater detail below, one purpose of the heat shrink material layer 40 relates to the manufacturing process of the sheath 10. More particularly, during manufacture, the shaft 12 is subjected to a heat treating process, such as, for example, a reflow process. During this process, the heat shrink layer 40 is caused to shrink when exposed to a suitable amount of heat. The heat applied to the shaft 12 also causes the polymeric material of the polymer tube 34 to melt, and the shrinking of the heat shrink layer 40 forces the polymeric material to flow into contact with the inner liner 24 and tubes 38 (in an embodiment of the sheath 10 that includes the tubes 38), as well as to flow into the braided wire assembly 36 of the shaft 12 (in an embodiment of the sheath 10 that includes the braided wire assembly 36). In an exemplary embodiment, the heat shrink material layer 40 remains as the outermost layer of the shaft 12. However, in another exemplary embodiment, the heat shrink material layer 40 is removed following the reflow process, and therefore, the polymer tube 34 is the outermost layer of the shaft 12. Accordingly, sheaths 10 that when fully assembled have a heat shrink material layer 40, and sheaths that when fully assembled do not have a heat shrink material layer 40, both remain within the spirit and scope of the present disclosure.

In an exemplary embodiment, the shaft 12 may further include a lubricious coating (not shown) that may cover the entire shaft 12 and the electrodes 14 mounted thereon, or just a portion thereof. In an exemplary embodiment, the coating 42 comprises siloxane. However, in other exemplary embodiments, the coating 42 may comprise one of any number of suitable hydrophilic coatings such as, for example, Hydromer® or Hydak® coatings. The purpose of the lubricious coating 42, which may be adjacent to either the polymer tube 34 or the heat shrink layer 40 (if the shaft 12 has a heat shrink layer 40), is to provide the shaft 12 with a smooth and slippery surface that is free of sharp edges, such that the shaft can move with ease when inserted into an anatomical structure.

As briefly described above, and as will be described in greater detail below, the sheath 10 includes one or more electrodes 14 mounted on the shaft 12. As illustrated in FIG. 1, the electrodes 14 may be disposed at or near the distal end 18 of the shaft 14, and may have a number of spacing configurations. In addition, or alternatively, one or more electrodes 14 may be disposed more proximally from the distal end 18. As will be described in greater detail below, in an exemplary embodiment, the shaft 12 is deflectable. In such an embodiment, the electrodes 14 may be mounted on deflectable portions of the shaft 12 and/or non-deflectable portions. In an exemplary embodiment, the electrodes 14 are flush with the outer surface of the shaft 12, and therefore, are recessed into the shaft 12.

The electrodes 14 may comprise any number of types of electrodes and may be used for any number of purposes. For example, the electrodes 14 may comprise one or more of magnetic coil(s), ring electrodes, tip electrodes, or a combination thereof. Further, the electrodes 14 may be used for a number of purposes or to perform one or more functions. For example, the electrodes 14 may be used in the pacing of the heart, monitoring electrocardiograph (ECG) signals, detecting location/position of the electrode 14 and therefore the sheath 10, mapping, visualization of the sheath 10, and the like. Additionally, one or more of the electrodes 14 may be formed of a radiopaque material, such as, for example and without limitation, a metallic material, such as, for example, platinum or another dense material. This permits the visualization of the electrodes 14 by an x-ray based visualization system, such as, for example, a fluoroscopic system. Further, the electrodes 14 may be low impedance electrodes (e.g., ≦600Ω).

In an embodiment wherein the sheath 10 includes the minor lumens 32 in the outer layer 26 of the shaft 12, each electrode 14 has one or more elongate lectrical conductors or wires 44 associated therewith and electrically coupled thereto. As described above, in such an embodiment, the sheath 10 includes one or more minor lumens 32 (i.e., 32₂, 32₄, 32₆, 32₈ in FIGS. 2 and 3) in the outer layer 26 of the shaft 12 configured to house, for example, the electrical wires 44 associated with the electrodes 14. In an exemplary embodiment, each minor lumen 32 configured to house an electrical wire 44 is configured to house the electrical wire 44 of a single corresponding electrode 14. Accordingly, the electrical wire 44 of a given electrode 14 is electrically connected to the electrode 14, passes through a portion of the outer layer 26 of the shaft 12, and is disposed within the corresponding minor lumen 32. When disposed within the minor lumens 32, the electrical wires 44 are permitted to move within the minor lumen 32 as the shaft 12 is deflected. The minor lumen 32 extends to the proximal end 16 of the shaft 12 such that the electrode wire 44 may be coupled to an interconnect or cable connector (not shown), which allows the electrode 14 to be coupled with other devices, such as a computer, a system for visualization, mapping and/or navigation, and the like. The interconnect is conventional in the art and is disposed at the proximal end 16 of the shaft 12.

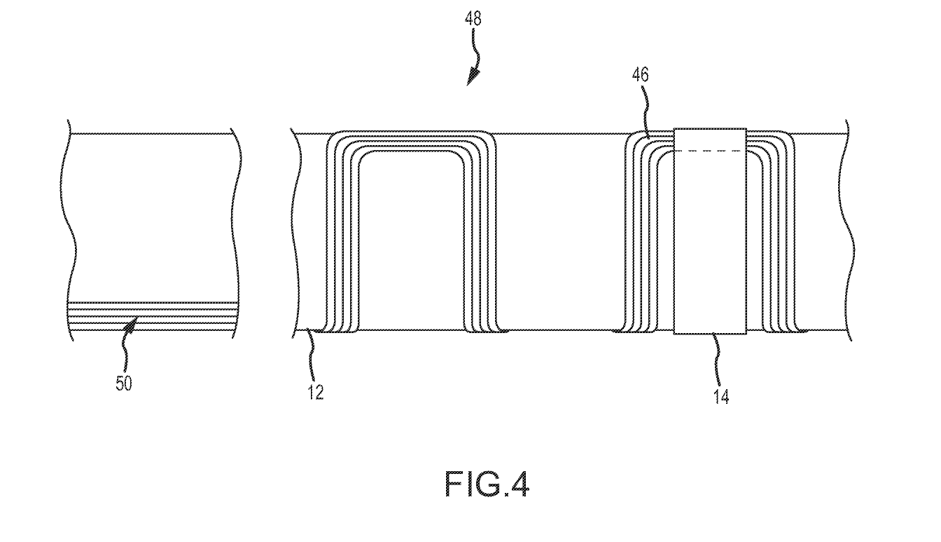

In another exemplary embodiment of the sheath 10 illustrated, for example, in FIG. 4, rather than the shaft 12, and the outer surface 26 thereof, in particular, having the minor lumens 32 for the electrical wires associated with the electrodes 14 disposed therein, a flexible circuit 46 comprising one or more electrical conductors is disposed within the outer surface 26. As with the minor lumens 32 described above, the flexible circuit 46 may extend from the proximal end 16 of the shaft 12 to the distal end 18. Alternatively, the flexible circuit 46 may extend from the proximal end 16 to the point on the shaft 12 at which the electrode(s) are mounted. The flexible circuit 46 is configured for electrical coupling with one or more of the electrodes 14. Accordingly, the number of electrical conductors in the flexible circuit 46 will at least equal the number of electrodes 14.

In an exemplary embodiment the flexible circuit 46 has two portions. A first portion 48 is disposed in a deflectable area on the shaft 12. In an exemplary embodiment, the first portion 48 of the flexible circuit 46 wraps around the shaft 12 in a serpentine pattern, and has one or more pads to which the electrodes 14 are electrically coupled. A second portion 50 of the flexible circuit 46 extends from the first portion 48 to the point at which the flexible circuit 46 terminates, such as, for example, at the proximal end 16 of the shaft 12. In an exemplary embodiment, the second portion 50 of the flexible circuit 46 is electrically coupled to an interconnect or connector (not shown), which allows the electrodes 14 to be coupled with other devices, such as a computer, a system for visualization, mapping and/or navigation, and the like. The interconnect is conventional in the art and is disposed at the proximal end 16 of the shaft 12.

It will be appreciated by those having ordinary skill in the art that but for the description relating to the minor lumens 32/tubes 38 being disposed within the outer layer 26 of the shaft 12, the description above relating to the construction and composition of the shaft 12 applies with equal force to an embodiment wherein the shaft 12 includes a flexible circuit 46 disposed therein. Accordingly, that disclosure will not be repeated, but rather is incorporated here by reference.

Whether the sheath 10 comprises minor lumens 32/tubes 38 or a flexible circuit 46 in the outer layer 26 of the shaft 12 thereof, in an exemplary embodiment, the sheath 10 may be steerable (i.e., the distal end 18 of the shaft 12 may be deflected in one or more directions relative to the longitudinal axis 22 of the sheath 10). In one exemplary embodiment, the movement of the sheath 10 may be controlled and operated manually by a physician. In another exemplary embodiment, however, movement of the sheath 10 may be controlled and operated by an automated guidance system, such as, for example and without limitation, a robotic-based system or a magnetic-based system.

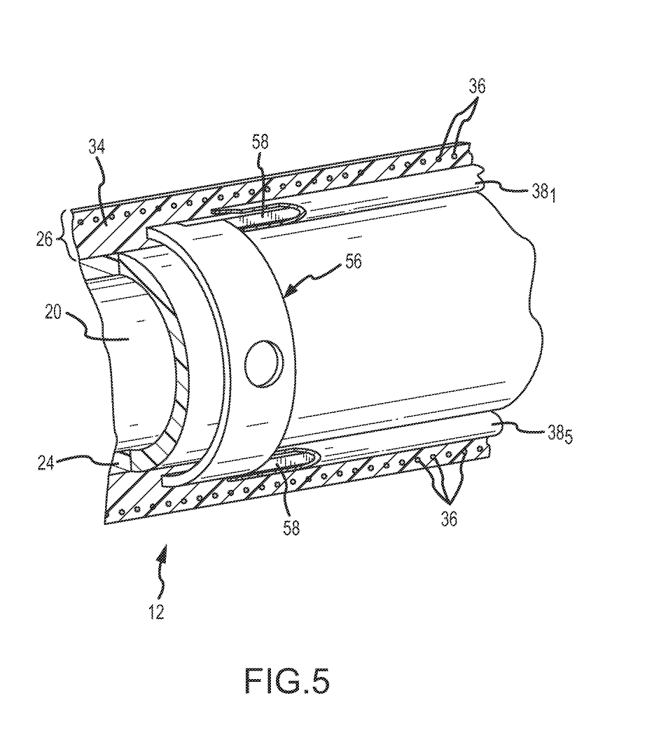

In an exemplary embodiment wherein the sheath 10 is configured for physician control, the sheath 10 includes a steering mechanism 52. A detailed description of an exemplary steering mechanism, such as steering mechanism 52, is set forth in U.S. Patent Publication No. 2007/0299424 entitled “Steerable Catheter Using Flat Pull Wires and Method of Making Same” filed on Dec. 29, 2006, the disclosure of which is hereby incorporated by reference in its entirety. Accordingly, with reference to FIGS. 1 and 5, the steering mechanism 52 will be briefly described. In an exemplary embodiment, the steering mechanism 52 comprises a handle 54, a pull ring 56 disposed in the shaft 12 of the sheath 10, and one or deflection elements, such as pull wires 58, coupled with both the handle 54 and the pull ring 56, and disposed within the shaft 12 of the sheath 10.

As illustrated in FIG. 1, the handle 54 is coupled to the shaft 12 at the proximal end 16 thereof. In an exemplary embodiment, the handle 54 provides a location for the physician/clinician to hold the sheath 10 and, in an exemplary embodiment, is operative to, among other things, effect movement (i.e., deflection) of the distal end 18 of the shaft 12 in one or more directions. The handle 54 is conventional in the art and it will be understood that the construction of the handle 54 may vary.

In an exemplary embodiment, the handle 54 includes an actuator 60 disposed thereon or in close proximity thereto, that is coupled to the pull wires 58 of the steering mechanism 52. The actuator 60 is configured to be selectively manipulated to cause the distal end 18 to deflect in one or more directions. More particularly, the manipulation of the actuator 60 causes the pull wires 58 to be pushed or pulled (the length of the pull wires is increased or decreased), thereby effecting movement of the pull ring 56, and thus, the shaft 12. The actuator 60 may take a number of forms known in the art. For example, the actuator 60 may comprise a rotatable actuator, as illustrated in FIG. 1, that causes the sheath 10, and the shaft 12 thereof, in particular, to be deflected in one direction when rotated one way, and to deflect in another direction when rotated in the other way. Additionally, the actuator 60 may control the extent to which the shaft 12 is able to deflect. For instance, the actuator 60 may allow the shaft 12 to deflect to create a soft curve of the shaft. Additionally, or in the alternative, the actuator 60 may allow the shaft 12 to deflect to create a more tight curve (e.g., the distal end 18 of the shaft 12 deflects 180 degrees relative to the shaft axis 22. It will be appreciated that while only a rotatable actuator is described in detail here, the actuator 60 may take on any form known the art that effects movement of the distal portion of a sheath or other medical device.

The actuator 60 is coupled to the pull wires 58 of the steering mechanism 52. In an exemplary embodiment, and as with the electrical wires 44 associated with the electrodes 14, the pull wires 58 are located within the outer layer 26 of the shaft 12. More particularly, the pull wires 58 are disposed within minor lumens 32 (i.e., lumens 32₁, 32₃, 32₅, 32₇ in FIGS. 2 and 3) in the outer layer 26, and are configured to extend from the handle 54 to the pull ring 56 (best shown in FIG. 5). In an exemplary embodiment, the pull wires 58 have a rectangular cross-section. In other exemplary embodiments, however, the pull wires 58 may have a cross-sectional shape other than rectangular, such as, for example and without limitation, a round or circular cross-sectional shape.

The steering mechanism 52 may comprise a number of different pull wire arrangements. For instance, in the exemplary embodiment illustrated in FIGS. 2 and 3, the steering mechanism 52 includes four pull wires 58. In this particular embodiment, the pull wires 58 are disposed 90 degrees apart from each other. In another exemplary embodiment, the steering mechanism comprises two pull wires 58. In such an embodiment, the pull wires 58 are spaced 180 degrees apart from each other.

In either embodiment, the minor lumens 32 within which the electrical wires 44 of the electrodes 14 are housed are located in between the minor lumens 32 for the pull wires 58, and along the neutral axis of the sheath 10. For example, in an exemplary embodiment, there are two pull wires 58, three electrical wires 44, and five minor lumens 32. In such an embodiment, the two minor lumens 32 with the pull wires 58 therein are disposed 180 degrees apart from each other. The remaining three minor lumens 32, each having an electrical wire 44 therein, are placed 90 degrees from each pull wire 58 (e.g., a pair of minor lumens 32 on one side, and one minor lumen 32 on the other). In another exemplary embodiment illustrated, for example, in FIGS. 2 and 3, there are four pull wires 58, four electrical wires 44, and eight minor lumens 32. In such an embodiment, the four minor lumens 32 with the pull wires 58 therein (i.e., lumens 32₁, 32₃, 32₅, 32₇ in FIGS. 2 and 3) are disposed 90 degrees apart from each other. The remaining four minor lumens 32, each having an electrical wire 44 therein (i.e., 32₂, 32₄, 32₆, 32₈ in FIGS. 2 and 3), are placed between each of the four pull wires 58.

The pull wires 58 are coupled at a first end to the actuator 60 and at the second end to the pull ring 56. FIG. 5 is a depiction of a portion of the shaft 12 having the outer layer 26 surrounding the pull ring 56 cut away. As illustrated in FIG. 5, the pull ring 56 is anchored to the shaft 12 at or near the distal end 18 thereof. One exemplary means by which the pull ring 56 is anchored is described in U.S. Patent Publication No. 2007/0199424 entitled “Steerable Catheter Using Flat Pull Wires and Method of Making Same” filed on Dec. 29, 2006, the entire disclosure of which was incorporated by reference above. Accordingly, as the pull wires 58 are pulled and/or pushed, the pull wires 58 pull and push the pull ring 56, thereby causing the shaft 12 to move (e.g., deflect). Accordingly, the physician manipulates the actuator 60 to cause the distal end 18 of the shaft 12 to move in a certain direction. The actuator 60 pulls and/or pushes the correct pull wires 58, which then causes the pull ring 56, and therefore the shaft 12, to move as directed.

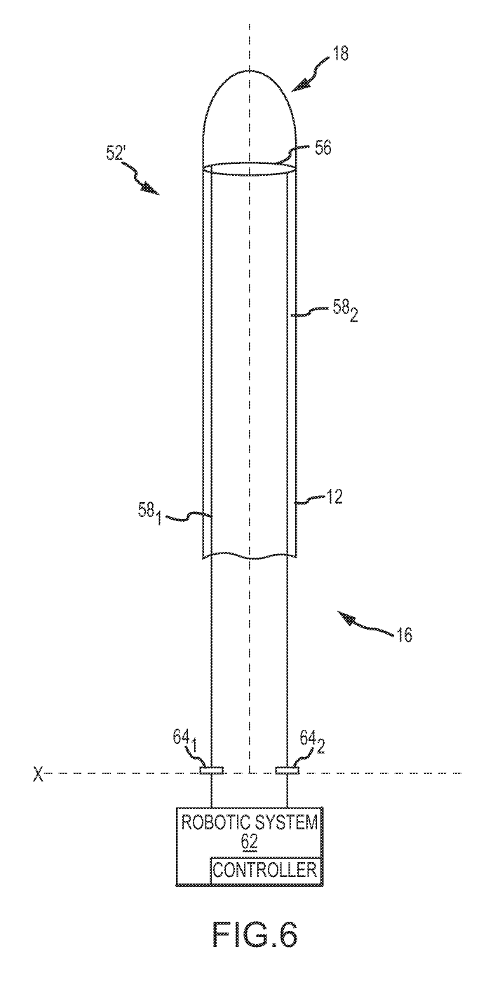

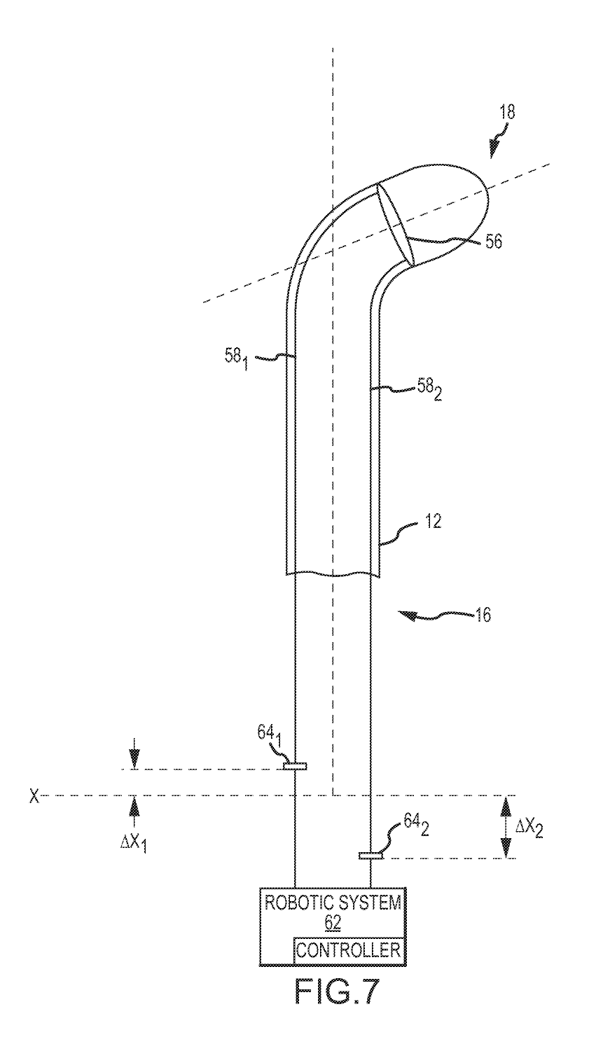

As briefly described above, in another exemplary embodiment, rather than being configured for manual control, the sheath 10 is controlled by an automated guidance system 62. With reference to FIGS. 6 and 7, in one exemplary embodiment the automated guidance system 62 is a robotic system (i.e., robotic system 62). In such an embodiment, the sheath 10 includes a steering mechanism 52′ that is coupled with the robotic system 62 and acts in concert with, and under the control of, the robotic system 62 to effect movement of the distal end 18 of the shaft 12. Detailed descriptions of exemplary arrangements/configurations by which a robotic system controls the movement of a medical device are set forth in PCT Patent Application Serial No. PCT/2009/038597 entitled “Robotic Catheter System with Dynamic Response” filed on Mar. 27, 2009 (International Publication No. WO/2009/120982), and U.S. Patent Publication No. 2009/0247993 entitled “Robotic Catheter System” filed on Dec. 31, 2008, the disclosures of which are hereby incorporated by reference in their entireties.

To summarize, in an exemplary embodiment, the steering mechanism 52′ comprises one or more pull wires 58 (i.e., 58₁ and 58₂ in FIGS. 6 and 7) and a pull ring 56. The description above with respect to these components applies here with equal force, and therefore, will not be repeated. However, unlike the embodiment described above, the steering mechanism 52′ further comprises one or more control members 64 (i.e., 64₁ and 64₂ in FIGS. 6 and 7) equal to the number of pull wires 58, and each control member 64 is affixed or coupled to a respective pull wire 58. The control members 64 are configured to interface or operatively connect control devices, such as, for example, motors or associated linkage or intermediate components thereof, to the pull wires 58. In such an embodiment, the control devices are controlled by a controller, which, in turn, may be fully automated and/or responsive to user inputs relating to the driving or steering of the sheath 10.

In either instance, movement of the control devices (e.g., movement of a motor shaft) is translated to cause one or more of the control members 64 to move, thereby resulting in the desired movement of the sheath 10, and the shaft 12 thereof, in particular. For example, FIG. 6 illustrates the shaft 12 in an undeflected state. Thus, both of the control members 64₁, 64₂ are co-located at a position X. However, FIG. 7 illustrated the shaft 12 in a deflected state. In this instance, the control member 64₁ has been pushed toward the distal end 18 of the shaft 12 a distance of ΔX₁, while the control member 64₂ has been pulled away from the distal end 18 of the shaft 12 a distance of ΔX₂. Accordingly, the robotic system 62 is configured to manipulate the positions of the control members 64 of the steering mechanism 52′ to effect movement of the shaft 12, and the distal end 18 thereof, in particular.

While the description of an automated sheath control system 62 has been with respect to one particular robotic system, other automated guidance systems and other types of robotic systems may be used. Accordingly, automated guidance systems other than robotic systems, and robotic-based automated guidance systems other than that described with particularity above, remain within the spirit and scope of the present disclosure.

It will be appreciated that in addition to the structure of the sheath 10 described above, another aspect of the present disclosure is a method of manufacturing a medical device, such as, for example, the sheath 10. As was noted above, the following description will be limited to an embodiment wherein the medical device is a sheath 10. It will be appreciated, however, that the methodology may be applied to medical devices other than a sheath, and therefore, those medical devices remain within the spirit and scope of the present disclosure.

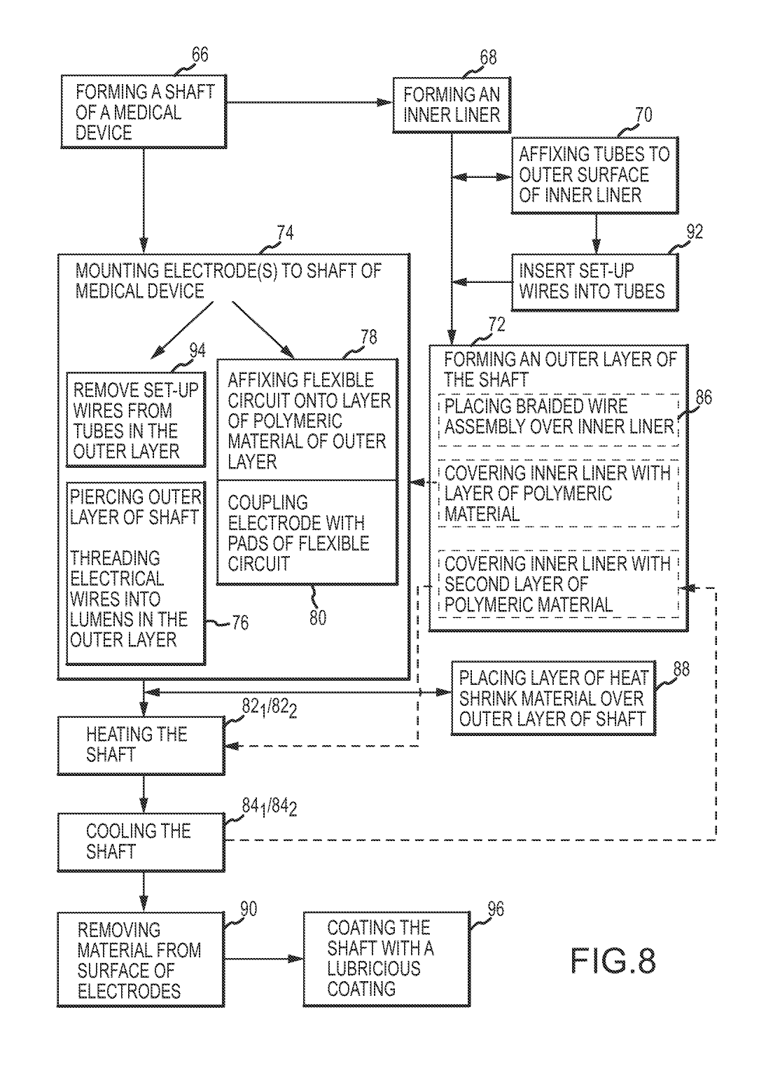

With reference to FIG. 8, in an exemplary embodiment, the method comprises a step 66 of forming a shaft of the sheath 10. The forming a shaft step 66 may comprise a number of substeps. In an exemplary embodiment, a substep 68 comprises forming an inner liner, such as, for example, the inner liner 24 described above. The inner liner 24 has a tubular shape, and has an inner surface 28 and an outer surface 30. In an exemplary embodiment, the inner liner 24 is formed by placing a liner material, such as, for example, etched PTFE, over a mandrel. In this embodiment, the mandrel is removed at or near the end of the manufacturing process, thereby resulting in the creation of the major lumen 20 in the inner liner 24. The inner liner 24 comprises the first layer of the shaft 12 of the sheath 10.

In an exemplary embodiment, the forming step 66 further includes a substep 70 of affixing one or more tubes, such as, for example, the tubes 38 described above, onto the outer surface 30 of the inner liner 24. Each tube 38 defines a minor lumen 32 therein in which, as was described above, a pull wire 58 or an electrical wire 44 is housed. The tubes 38 may be affixed to the outer surface 30 in a number of ways. In an exemplary embodiment, the tubes 38 are affixed using an adhesive, such as, for example, cyanoacrylate.

The forming step 66 still further comprise a substep 72 of forming on outer layer of the shaft 12, such as, for example, the outer layer 26 described above. In an exemplary embodiment, substep 72 comprises covering the inner liner 24 and the tube(s) 38 affixed thereto, if applicable, with one or more layers of polymeric material to form the outer layer 26. For example, in an exemplary embodiment that will be described in greater detail below, the outer layer 26 is formed of two layers of polymeric material. In such an embodiment, the inner liner 24 may be covered with a first layer or tube 34 of polymeric material, and then a second layer or tube 34 of polymeric material. In an exemplary embodiment, the second layer of polymeric material is applied after one or more electrodes 14 are mounted onto the shaft 12. The substep 72 may comprise placing one or more tubes formed of a polymeric material, such as the tube 34 described above, over the inner liner 24.

The method yet still further comprises a step 74 of mounting one or more electrodes 14 onto the shaft 12, and onto a layer of polymeric material, in particular. It may be desirable that the sheath 10, and the shaft 12 thereof, in particular, be smooth and free of sharp edges. Accordingly, the mounting step 74 may comprise recessing the electrode(s) into the outer layer 26. In an exemplary embodiment, this is done by swaging the outer surface of the electrodes 14 down, thereby forcing the bottom or inner surface of the electrodes 14 down and locking the electrodes 14 into place.

In an exemplary embodiment, the electrodes 14 may be mounted to the outer surface of the outer layer 26. However, in as described above, in an exemplary embodiment, the electrodes 14 are mounted to the shaft 12 after the inner liner 24 is covered with a first layer or tube of polymeric material, and before the inner liner 24 is covered with a second layer or tube of polymeric material. Accordingly, in such an embodiment the electrodes are mounted prior to the completion of the substep 72 of forming the outer layer 26 of the shaft 12.

In an exemplary embodiment wherein the shaft 12 includes one or more minor lumens 32 therein for housing electrical wires 44 associated with the electrodes 14, the mounting step 74 comprises a substep 76 of threading the electrical wires 44 associated with the electrodes into the corresponding minor lumens 32. Accordingly, the substep 76 is performed for each electrode 14 being mounted to the shaft 12. In an exemplary embodiment, the substep 76 comprises piercing or puncturing the outer layer 26 of the shaft 12 at the location at which the electrode 14 is to be mounted to provide access to the distal end of the corresponding minor lumen 32. The electrical wire 44 associated with the electrode 14 is then threaded through the hole in the outer layer 26 and into the minor lumen 32. The electrical wire 44 is then advanced down the minor lumen 32 to the proximal end thereof where the electrical wire 44 may be coupled to an interconnect or connector, such as, for example, the interconnect described above. As the electrical wire 44 is advanced down the minor lumen 32, the electrode 14 is pulled into place on the shaft 12 and covers and seals the access hole through which the electrical wire 44 was inserted. This process is then repeated for each electrode 14 being mounted on the shaft 12. As described above, in an exemplary embodiment, once all of the electrodes are mounted to the shaft 12, the shaft 12 and the electrodes 14 are covered with a layer of polymeric material (i.e., a second layer of polymeric material for the outer layer 26), such as, for example, a polymer tube 34, as part of the substep 72 of forming the outer layer 26.

In another exemplary embodiment, rather than having minor lumens 32 therein for housing electrical wires 44, a flexible circuit, such as, for example, the flexible circuit 46 described above, is disposed within the outer layer 26 of the shaft 12. In an exemplary embodiment, the placement of the flexible circuit 46 within the shaft 12, and the outer layer 26 thereof, in particular, is performed as part of the mounting step 74 and before the completion of the formation of the outer layer 26. In such an embodiment, the mounting step 74 comprises a substep 78 of affixing the flexible circuit 46 to the first layer of polymeric material that covers the inner liner 24. In this embodiment, the mounting step 74 further comprises a second substep 80 of electrically coupling each electrode to a corresponding electrode pad of the flexible circuit 46. In an exemplary embodiment, the electrodes 14 are crimped onto the pads of the flexible circuit 46. This process is then repeated for each electrode 14 being mounted on the shaft 12. As described above with respect to the embodiment of the sheath 10 comprising the tubes 38, in an exemplary embodiment, once all of the electrodes 14 are mounted to the shaft 12, the shaft 12 and the electrodes 14 are covered with a layer of polymeric material, such as, for example, a polymer tube 34, as part of the substep 72 of forming the outer layer 26.

In an exemplary embodiment, the method further comprises performing one or more heat treating processes, such as, for example, a reflow process, on at least a portion of the shaft 12, and the outer layer 26 thereof, in particular. Accordingly, in one such embodiment, the method comprises a step 82 of heating the shaft 12 to a temperature at which the polymeric material thereof melts and redistributes around the circumference of the shaft 12. In one exemplary embodiment, the temperature applied to the shaft 12 is 400 degrees (F.) and the rate of exposure is 1 cm/minute. It will be appreciated, however, that temperature and the rate of exposure may vary depending on various factors, such as, for example, the material used. Accordingly, the present invention is not meant to be limited to the specific temperature and rate set forth above, and other temperatures and rates remain within the spirit and scope of the present disclosure.

In an exemplary embodiment, multiple heating steps are performed on the shaft 12 at multiple points in the manufacturing process. For example, in the embodiments described above wherein the outer layer 26 comprises two layers of polymeric material, two heating processes are performed. More particularly, after the inner liner 24 is covered with the first layer or tube 34, a first heating step 82₁ is performed. After the application of a second layer or tube 34 over said inner liner 24, a second heating step 82₂ is performed.

Once the heating step 82 is complete, a step 84 of cooling the shaft 12, and therefore, the polymeric material, is performed. In an exemplary embodiment, the cooling step 84 comprises letting the shaft 12 air-cool. However, in another exemplary embodiment, a cooling process may be performed on the shaft 12.

As with the heating step described above, in an exemplary embodiment, multiple cooling steps are performed on the shaft 12 at multiple points in the manufacturing process. For instance, in an embodiment wherein the outer layer 26 comprises two layers of polymeric material or tubes 34, a first cooling step 84₁ is performed after the first layer or tube 34 is heated. After the second layer or tube 34 is heated, a second cooling step 84₂ is performed.

In an exemplary embodiment, prior to covering the inner liner 24 with polymeric material, the forming an outer layer of the shaft substep 72 further comprises a substep 86 of placing a braided wire assembly, such as the braided wire assembly 36 described above, over the inner liner 24 and the tubes 38, if applicable. In such an embodiment, once the substep 86 is complete, the substep(s) of covering the inner liner 24 with a polymeric material is performed. Therefore, the combination of the braided wire assembly 36 and the polymeric material comprises the outer layer 26.

In an exemplary embodiment, and prior to performing the heating step 82, the method further comprises a step 88 of placing a layer of heat shrink material, such as, for example, the heat shrink material layer 40 described above, over the outer layer 26 of the shaft 12. The heat shrink material layer 40 is formed of a material that has a higher melt temperature than that of the polymeric material of the outer layer 26 such that when the heating step 82 is performed, the heat shrink material layer 40 retains it tubular shape and forces the polymeric material into the braided wire assembly 36 (if the shaft 12 comprises a braided wire assembly 36), and into contact with the inner liner 24, tubes 38, and/or flexible circuit 46 (depending on the construction and composition of the shaft 12), but does not itself melt. In an exemplary embodiment, following the heating step 82 and either during or following the cooling step 84, the heat shrink material layer 40 is removed. Alternatively, the heat shrink material layer 40 is not removed, but rather remains as part of the shaft 12.

In certain embodiments, the electrodes 14 may be covered with one or more layers of material, such as, for example, polymeric material or heat shrink material. This may be because the electrodes 14 were covered with a layer of polymeric material during the formation of the outer layer 26, or because polymeric material migrated onto the surface of the electrodes 14 during a heating process performed on the shaft 12. In either instance, the method further comprises a step 90 of removing the material from the outer surface of the electrodes 14. Step 90 may be performed in a number of ways, such as, for exemplary purposes only, laser ablating the material away from the surface of the electrodes 14. It will be appreciated by those having ordinary skill in the art, however, that other known processes or techniques may be used to remove the material, and those processes or techniques remain within the spirit and scope of the present disclosure.

In addition to the description above, in an embodiment wherein the shaft 12 includes the minor lumens 32 therein, the method may further comprise a step 92 of inserting set-up wires into one or more of the minor lumens 32 defined by the tubes 38. The purpose of inserting set-up wires in the minor lumens 32 is to prevent the tubes 38 from collapsing during the subsequent steps of the manufacturing process. Accordingly, either prior to tubes 38 being affixed to the outer surface 30 of the inner liner 24 or after the tubes 38 are affixed, set-up wires are inserted into the minor lumens 32. Following the performance of one or more heat treating processes on the shaft 12, in a step 94, the set-up wires are removed from the minor lumens 32 and replaced with the electrical wires 44.

In an exemplary embodiment, following the cooling step 84 and/or the removal step 90, the method further comprises a step 96 of coating the outer surface of the shaft 12, and in an exemplary embodiment the outer surface of the electrodes 14 as well, with a lubricious coating, such as, for example, the lubricious coating described above.

In accordance with another aspect of the disclosure, the sheath 10 is part of a system 98 for performing one or more diagnostic or therapeutic medical procedures, such as, for example and without limitation, drug delivery, the pacing of the heart, pacer lead placement, tissue ablation, monitoring, recording, and/or mapping of electrocardiograph (ECG) signals and other electrophysiological data, and the like. In addition to the sheath 10, the system 98 comprises, at least in part, a system 100 for visualization, mapping, and/or navigation of internal body structures and medical devices. In an exemplary embodiment, the system 100 includes an electronic control unit (ECU) 102 and a display device 104. In another exemplary embodiment, the display device 104 is separate and distinct from the system 100, but electrically connected to and configured for communication with the ECU 102.

As will be described in greater detail below, one purpose of the system 100 is to accurately determine the position and orientation of the sheath 10, and in certain embodiments, to accurately display the position and orientation of the sheath 10 for the user to see. Knowing the position and orientation of the sheath 10 is beneficial regardless of whether the sheath is manually controlled (i.e., by a physician or clinician) or controlled by an automated guidance system, such as, for example, a robotic-based or magnetic-based system. For example, in a robotic-based system, it is important to know the accurate position and orientation of the sheath 10 to minimize error and provide patient safety by preventing perforations to the cardiac tissue. In a magnetic-based systems, it is important for the physician/clinician operating the system to know the accurate location and orientation of, for example, the fulcrum of a catheter used with the sheath 10. This information allows the physician/clinician to direct the orientation of the sheath 10 to optimize the ability to locate the catheter precisely and take full advantage of the magnetic manipulation capability offered by magnetic-based systems.

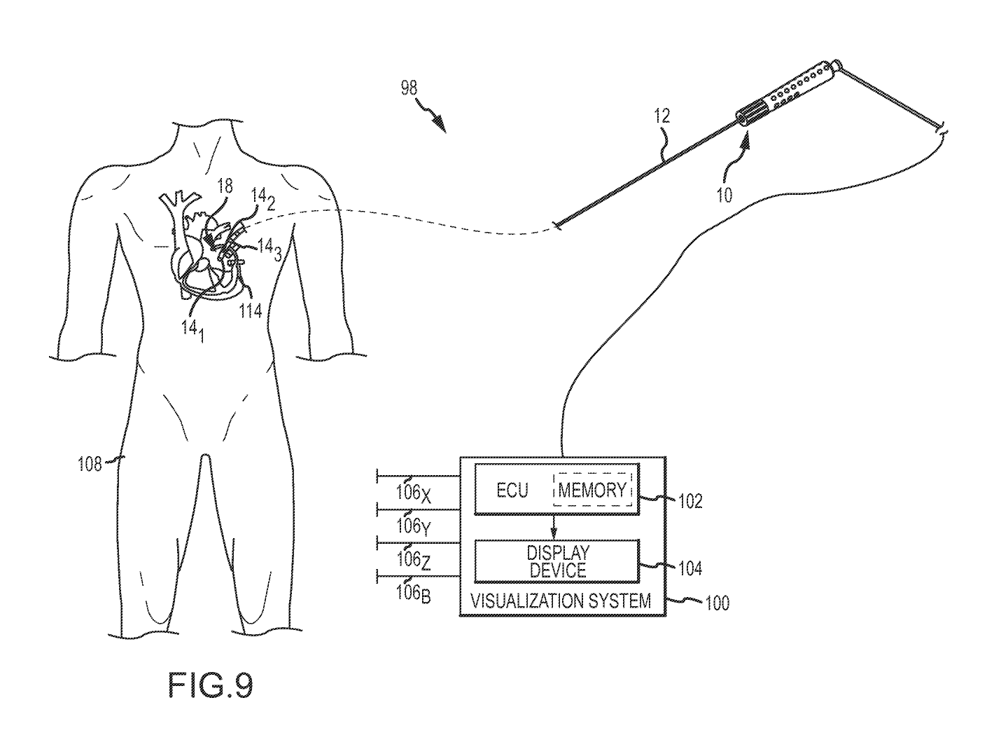

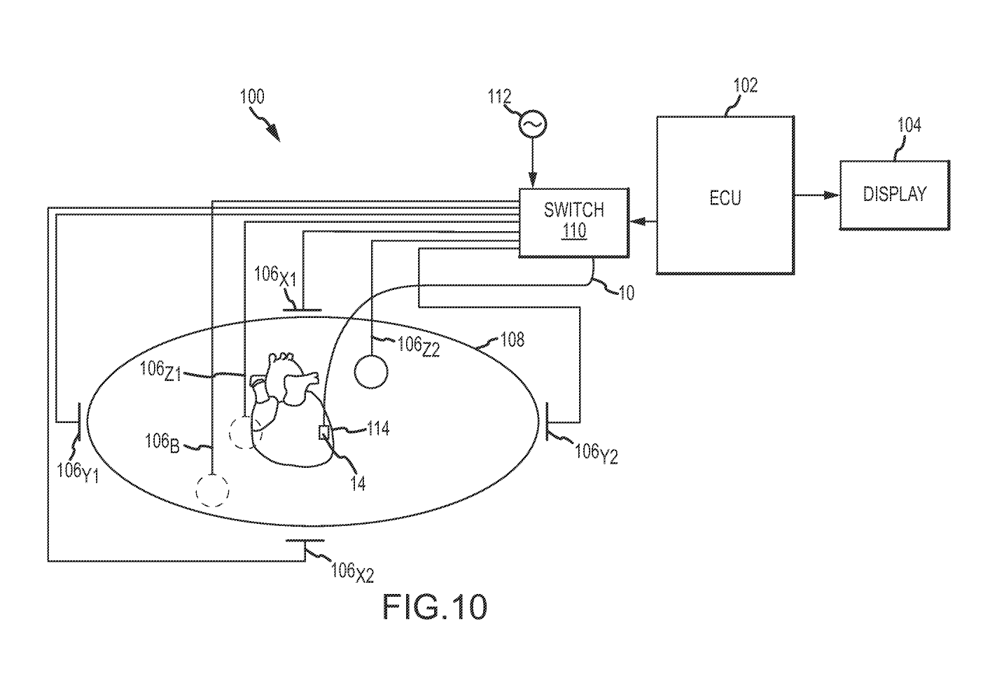

With reference to FIGS. 9 and 10, the visualization, navigation, and/or mapping system 100 will be described. The system 100 may comprise an electric field-based system, such as, for example, the EnSite NavX™ system commercially available from St. Jude Medical, Inc., and as generally shown with reference to U.S. Pat. No. 7,263,397 entitled “Method and Apparatus for Catheter Navigation and Location and Mapping in the Heart,” the disclosure of which is incorporated herein by reference in its entirety. In other exemplary embodiments, however, the system 100 may comprise systems other than electric field-based systems. For example, the system 100 may comprise a magnetic field-based system such as the Carto™ system commercially available from Biosense Webster, and as generally shown with reference to one or more of U.S. Pat. No. 6,498,944 entitled “Intrabody Measurement;” U.S. Pat. No. 6,788,967 entitled “Medical Diagnosis, Treatment and Imaging Systems;” and U.S. Pat. No. 6,690,963 entitled “System and Method for Determining the Location and Orientation of an Invasive Medical Instrument,” the disclosures of which are incorporated herein by reference in their entireties. In another exemplary embodiment, the system 100 comprises a magnetic field-based system such as the gMPS system commercially available from MediGuide Ltd., and as generally shown with reference to one or more of U.S. Pat. Nos. 6,233,476 entitled “Medical Positioning System;” U.S. Pat. No. 7,197,354 entitled “System for Determining the Position and Orientation of a Catheter;” and U.S. Pat. No. 7,386,339 entitled “Medical Imaging and Navigation System,” the disclosures of which are incorporated herein by reference in their entireties. In yet another embodiment, the system 100 may comprise a combination electric field-based and magnetic field-based system, such as, for example and without limitation, the Carto 3™ system also commercially available from Biosense Webster, and as generally shown with reference to U.S. Pat. No. 7,536,218 entitled “Hybrid Magnetic-Based and Impedance Based Position Sensing,” the disclosure of which is incorporated herein by reference in its entirety. In yet still other exemplary embodiments, the system 100 may comprise or be used in conjunction with other commonly available systems, such as, for example and without limitation, fluoroscopic, computed tomography (CT), and magnetic resonance imaging (MRI)-based systems. For purposes of clarity and illustration only, the system 100 will be described hereinafter as comprising an electric field-based system.

As illustrated in FIGS. 9 and 10, in addition to the ECU 102 and the display 104, in an exemplary embodiment the system 100 further comprises a plurality of patch electrodes 106. With the exception of the patch electrode 106_{B} called a “belly patch,” the patch electrodes 106 are provided to generate electrical signals used, for example, in determining the position and orientation of the sheath 10, and potentially in the guidance thereof. In one embodiment, the patch electrodes 106 are placed orthogonally on the surface of a patient's body 108 and used to create axes-specific electric fields within the body 108. For instance, in one exemplary embodiment, the patch electrodes 106ₓ₁, 106ₓ₂ may be placed along a first (x) axis. The patch electrodes 106_{y1}, 106_{y2} may be placed along a second (y) axis. Finally, the patch electrodes 106_{z1}, 106ₓ₂ may be placed along a third (z) axis. Each of the patch electrodes 106 may be coupled to a multiplex switch 110. In an exemplary embodiment, the ECU 102 is configured through appropriate software to provide control signals to switch 110 to thereby sequentially couple pairs of electrodes 106 to a signal generator 112. Excitation of each pair of electrodes 106 generates an electric field within the body 108 and within an area of interest such as, for example, heart tissue 114. Voltage levels at non-excited electrodes 106, which are referenced to the belly patch 106_{B}, are filtered and converted, and provided to the ECU 102 for use as reference values.

As described above, the sheath 10 includes one or more electrodes 14 mounted thereon. In an exemplary embodiment, one of the electrodes 14 is a positioning electrode (however, in another exemplary embodiment, a plurality of the electrodes 14 are positioning electrodes). The positioning electrode 14 may comprise, for example and without limitation, a ring electrode or a magnetic coil sensor. The positioning electrode 14 is placed within electric fields created in the body 108 (e.g., within the heart) by exciting patch electrodes 106. The positioning electrode 14 experiences voltages that are dependent on the location between the patch electrodes 106 and the position of the positioning electrode 14 relative to the heart tissue 114. Voltage measurement comparisons made between the electrode 14 and the patch electrodes 106 can be used to determine the position of the positioning electrode 14 relative to the heart tissue 114. Movement of the positioning electrode 14 proximate the heart tissue 114 (e.g., within a heart chamber, for example) produces information regarding the geometry of the tissue 114. This information may be used, for example and without limitation, to generate models and maps of tissue or anatomical structures. Information received from the positioning electrode 14 (or if multiple positioning electrodes, the positioning electrodes 14) can be used to display on a display device, such as display device 104, the location and orientation of the positioning electrode 14 and/or the distal end of the sheath 10, and the shaft 12 thereof, in particular, relative to the tissue 114. Accordingly, among other things, the ECU 102 of the system 100 provides a means for generating display signals used to control the display device 104 and the creation of a graphical user interface (GUI) on the display device 104.

Accordingly, the ECU 102 may provide a means for determining the geometry of the tissue 114, EP characteristics of the tissue 114, and the position and orientation of the sheath 10. The ECU 102 may further provide a means for controlling various components of the system 100, including, without limitation, the switch 110. It should be noted that while in an exemplary embodiment the ECU 102 is configured to perform some or all of the functionality described above and below, in another exemplary embodiment, the ECU 102 may be a separate and distinct component from the system 100, and the system 100 may have another processor configured to perform some or all of the functionality (e.g., acquiring the position/location of the positioning electrode/sheath, for example). In such an embodiment, the processor of the system 100 would be electrically coupled to, and configured for communication with, the ECU 102. For purposes of clarity only, the description below will be limited to an embodiment wherein the ECU 102 is part of the system 100 and configured to perform all of the functionality described herein.

The ECU 102 may comprise a programmable microprocessor or microcontroller, or may comprise an application specific integrated circuit (ASIC). The ECU 102 may include a central processing unit (CPU) and an input/output (I/O) interface through which the ECU 102 may receive a plurality of input signals including, for example, signals generated by patch electrodes 106 and the positioning electrode 14, and generate a plurality of output signals including, for example, those used to control and/or provide data to the display device 104 and the switch 110. The ECU 102 may be configured to perform various functions, such as those described in greater detail below, with appropriate programming instructions or code (i.e., software). Accordingly, the ECU 102 is programmed with one or more computer programs encoded on a computer storage medium for performing the functionality described herein.

In operation, the ECU 102 generates signals to control the switch 110 to thereby selectively energize the patch electrodes 106. The ECU 102 receives position signals (location information) from the sheath 10 (and particularly the positioning electrode 14) reflecting changes in voltage levels on the positioning electrode 14 and from the non-energized patch electrodes 106. The ECU 102 uses the raw location data produced by the patch electrodes 106 and positioning electrode 14 and corrects the data to account for respiration, cardiac activity, and other artifacts using known or hereinafter developed techniques. The ECU 102 may then generate display signals to create an image or representation of the sheath 10 that may be superimposed on an EP map of the tissue 114 generated or acquired by the ECU 102, or another image or model of the tissue 114 generated or acquired by the ECU 102.

In an embodiment wherein there are multiple positioning electrodes 14, the ECU 102 may be configured to receive positioning signals from two or more of the positioning electrodes 14, and to then create a representation of the profile of the distal portion of the sheath 10, for example, that may be superimposed onto an EP map of the tissue 114 generated or acquired by the ECU 102, or another image or model of the tissue 114 generated or acquired by the ECU 102.

One example where this functionality is valuable relates to the treatment of atrial fibrillation. In atrial fibrillation, often the left side of the heart has to be accessed. Using a technique called transseptal access, the physician uses a long, small diameter needle to pierce or puncture the heart's septal wall in an area known as the fossa ovalis to provide a means of access from the right atrium to the left atrium. Once transseptal access is obtained, physicians prefer not to lose it. However, for a variety of reasons, there are times when the access to the left side through the fossa ovalis is lost. As a result, the procedure time is increased and additional piercing or puncturing of the septal wall may be required.

If multiple positioning electrodes are mounted on the sheath, however, using the system 102 the location of the positioning electrodes 14, and therefore, the sheath 10 can be determined, and a shadow representation of the sheath 10 may be superimposed onto an image or model of the tissue 114 showing its position across the fossa ovalis. This gives the physician a reference to use as guidance, and more particularly, permits the physician to reposition the sheath 10 in the same location as the shadow representation, should access to the left side be lost during the procedure. Thus, additional piercing or puncturing of the septal wall may be avoided, the speed of the procedure will be reduced, and fluoroscopy time may also be reduced. Further, the positioning electrodes 14 can be used in real time to “straddle” the fossa ovalis so as to allow the physician to try to prevent the sheath 10 from coming out of the fossa ovalis in the first place.

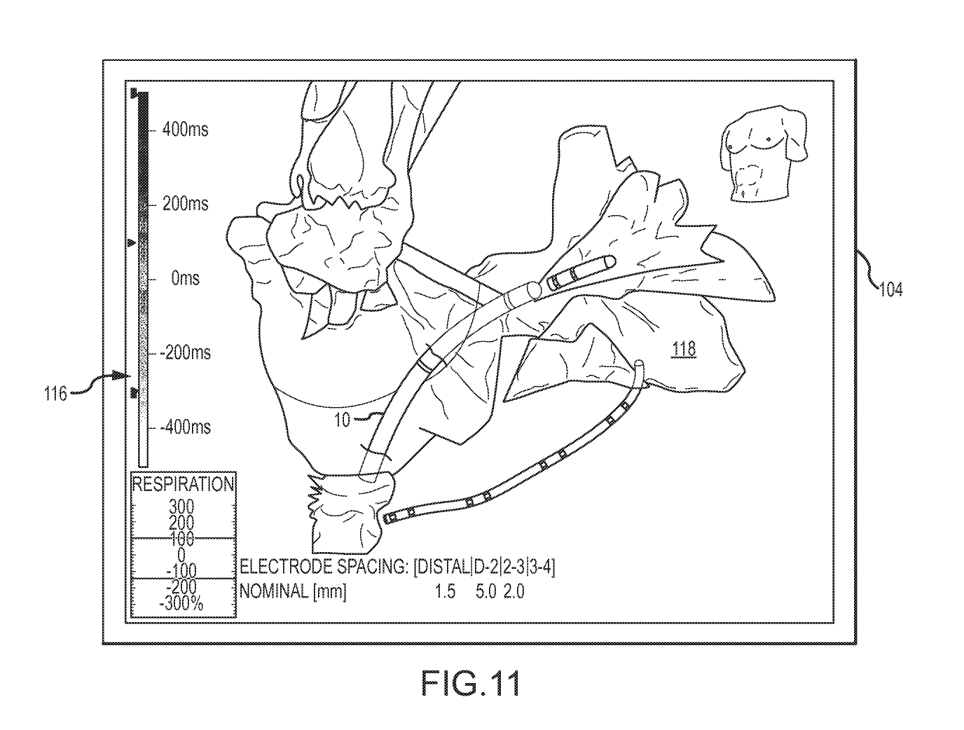

With reference to FIGS. 9 and 11, the display device 104, which, as described above, may be part of the system 100 or a separate and distinct component, is provided to convey information to a clinician to assist in, for example, the performance of therapeutic or diagnostic procedures on the tissue 114. The display device 104 may comprise a conventional computer monitor or other display device known in the art. With particular reference to FIG. 11, the display device 104 presents a graphical user interface (GUI) 116 to the clinician. The GUI 116 may include a variety of information including, for example and without limitation, an image or model of the geometry of the tissue 114, EP data associated with the tissue 114, electrocardiograms, electrocardiographic maps, and images or representations of the sheath 10 and/or positioning electrode 14. Some or all of this information may be displayed separately (i.e., on separate screens), or simultaneously on the same screen. The GUI 116 may further provide a means by which a clinician may input information or selections relating to various features of the system 100 into the ECU 102.

The image or model of the geometry of the tissue 114 (image/model 118 shown in FIG. 11) may comprise a two-dimensional image of the tissue 108 (e.g., a cross-section of the heart) or a three-dimensional image of the tissue 114. The image or model 118 may be generated by the ECU 102 of the system 100, or alternatively, may be generated by another imaging, modeling, or visualization system (e.g., fluoroscopic, computed tomography (CT), magnetic resonance imaging (MRI), etc. based systems) that are communicated to, and therefore, acquired by, the ECU 102. As briefly mentioned above, the display device 104 may also include an image or representation of the sheath 10 and/or the positioning electrode 14 illustrating their position and orientation relative to the tissue 114. The image or representation of the sheath 10 may be part of the image 118 itself (as is the case when, for example, a fluoroscopic system is used) or may be superimposed onto the image/model 118.

It will be appreciated that as briefly described above, in an exemplary embodiment, one or more of the electrodes 14 mounted on the shaft 12 may be used for purposes other than for determining positioning information. For example, one or more electrodes may be used for pacing in the atrium of the heart to, for example, determine bi-directional block on the septal wall.

In addition, or alternatively, one or more of the electrodes 14 may be used for monitoring electrocardiographs or to collect EP data in one or more areas in the heart. The information or data represented by the signals acquired by these electrodes 14 may be stored by the ECU 102 (e.g., in a memory of the device, for example), and/or the ECU 102 may display the data on an EP map or another image/model generated or acquired by the ECU 102, or otherwise display the data represented by the signals acquired by the electrodes 14 on a display device such as, for example, the display device 104. For example, in an exemplary embodiment, one or more electrodes 14 may be positioned such that as a therapeutic procedure is being performed on the left side of the fossa ovalis, ECGs or other EP data may be monitored on both the left and right sides of the fossa ovalis using the electrodes 14. One benefit of such an arrangement is that fewer medical devices need to be used during a procedure.

Accordingly, the system 98, and the visualization, navigation, and/or mapping system 100 thereof, in particular, is configured to carry out and perform any number of different functions, all of which remain within the spirit and scope of the present invention.

It should be understood that the system 100, and particularly the ECU 102 as described above, may include conventional processing apparatus known in the art, capable of executing pre-programmed instructions stored in an associated memory, all performing in accordance with the functionality described herein. It is contemplated that the methods described herein, including without limitation the method steps of embodiments of the invention, will be programmed in a preferred embodiment, with the resulting software being stored in an associated memory and where so described, may also constitute the means for performing such methods. Implementation of the invention, in software, in view of the foregoing enabling description, would require no more than routine application of programming skills by one of ordinary skill in the art. Such a system may further be of the type having both ROM, RAM, a combination of non-volatile and volatile (modifiable) memory so that the software can be stored and yet allow storage and processing of dynamically produced data and/or signals.

Although only certain embodiments have been described above with a certain degree of particularity, those skilled in the art could make numerous alterations to the disclosed embodiments without departing from the scope of this disclosure. Joinder references (e.g., attached, coupled, connected, and the like) are to be construed broadly and may include intermediate members between a connection of elements and relative movement between elements. As such, joinder references do not necessarily infer that two elements are directly connected/coupled and in fixed relation to each other. Additionally, the terms electrically connected and in communication are meant to be construed broadly to encompass both wired and wireless connections and communications. It is intended that all matter contained in the above description or shown in the accompanying drawings shall be interpreted as illustrative only and not limiting. Changes in detail or structure may be made without departing from the invention as defined in the appended claims.

1. A deflectable medical device, comprising
a shaft having a proximal end, a distal end, and a major lumen disposed therein extending between said proximal and said distal ends and configured to receive a second medical device therein, said shaft further comprising
an inner liner having an inner surface and an outer surface, said inner surface forming said major lumen; and
an outer layer comprising a polymeric material adjacent said outer surface of said inner liner;
means for deflecting said shaft in at least one direction relative to a longitudinal axis of said shaft coupled to a proximal portion of said shaft;
an electrode mounted on said shaft; and
a minor lumen disposed between the inner surface of the inner liner and an outer surface of the outer layer and in which an electrical wire of said electrode is disposed, said minor lumen extending from said proximal end of said shaft to a location on said shaft near where said electrode is mounted.

2. (canceled)

3. The medical device of claim 1 wherein said minor lumen comprises a spaghetti tube disposed within said outer layer, said spaghetti tube adapted to receive an elongate conductor coupled to said electrode.

4. The medical device of claim 1, wherein said outer layer has a flexible circuit disposed therein, said flexible circuit electrically coupled to said electrode.

5. The medical device of claim 1, wherein said outer layer further comprises a braided wire assembly, wherein said braided wire assembly includes at least one of a flat wire braid and a round wire braid.

6. The medical device of claim 1, wherein at least a portion of said outer layer of said shaft comprises one of a transparent and a translucent polymeric material.

7. The medical device of claim 1, wherein said means for deflecting said shaft comprises four deflectable elements disposed within said outer layer of said shaft and offset from each other by 90 degrees.

8. The medical device of claim 1, wherein said outer layer of said shaft comprises a plurality of portions having different durometers.

9. A method of manufacturing a medical device, said method comprising the steps of:
forming a shaft of said medical device, said forming a shaft step comprising the substeps of:
forming an inner liner having a tubular shape, wherein said inner liner has an inner surface and an outer surface; and
forming an outer layer by covering said inner liner with a polymeric material;
mounting an electrode onto said shaft;
heating said shaft to a predetermined temperature at which said polymeric material melts; and
cooling said shaft.

10. The method of claim 9, wherein said forming a shaft step further comprises the substep of affixing a tube onto said outer surface of said inner liner prior to said forming an outer layer step, said tube comprising a minor lumen.

11. The method of claim 9, wherein said forming an outer layer step further comprises the substep of placing a braided wire assembly over said inner liner prior to said substep of covering said inner liner with a polymeric material.

12. The method of claim 9 further comprising the steps of:
piercing a hole in said outer layer; and
inserting an electrode wire connected to said electrode through said hole in said outer layer and into said minor lumen.

13. The method of claim 9 further comprising the steps of:
inserting a set-up wire into said minor lumen prior to said step of heating said polymeric material; and
removing said set-up wire from said minor lumen following said step of heating said polymeric material.

14. The method of claim 9 further comprising the step of removing material covering the outer surface of said electrode following said heating step.

15. The method of claim 9, wherein said polymeric material covering said inner liner comprises a first layer of polymeric material and said heating and cooling steps comprise second heating and cooling steps, said forming an outer layer substep further comprising:
performing a first heating step on said shaft to heat said first layer of p polymeric material to a predetermined temperature at which said first layer of polymeric material melts;
performing a second cooling step on said shaft to cool said shaft; and
applying a second layer of polymeric material over said first layer of polymeric material following said mounting of an electrode step;
and further wherein said second heating step comprises heating said second layer of polymeric material, and said second cooling said comprises cooling said shaft following said second heating step.

16. The method of claim 15, wherein:
said forming a shaft step further comprises the substep of affixing a tube onto said outer surface of said inner liner prior to covering said inner liner with said first layer of polymeric material, said tube comprising a minor lumen; and
said mounting an electrode step comprises:
piercing said first layer of polymeric material to form a hole;
threading an electrical wire of said electrode through said hole and into said minor lumen;
covering said hole with said electrode.

17. The method of claim 15, wherein said mounting step comprises:
affixing a flexible circuit to said first layer of polymeric material; and
electrically coupling said electrode with said flexible circuit.

18. A system for performing at least one of a therapeutic and diagnostic medical procedure, comprising:
a first medical device having an elongate shaft and at least one electrode mounted on said shaft, said shaft comprising:
a proximal end, a distal end, and a major lumen therein extending between said proximal and distal ends and sized so as to receive a second medical device therein;
an inner liner having an inner surface and an outer surface, said inner liner surface forming said major lumen; and
an outer layer adjacent said outer surface of said inner liner,
a minor lumen disposed between the inner surface of the inner liner and an outer surface of the outer layer and in which an electrical wire of said electrode is disposed, said minor lumen extending from said proximal end of said shaft to a location on said shaft near where said electrode is mounted; and
an electronic control unit (ECU) configured to receive signals from said at least one electrode and, in response to said received signals, automatically perform at least one of:
determining a position of said at least one electrode; and
monitoring electrophysiological data.

19. The system of claim 18, wherein said ECU is configured to generate a representation of said first medical devices, said ECU further configured to superimpose said representation of said first medical device onto one of a model of an anatomical structure and an image of said anatomical structure.

20. The system of claim 18, wherein said ECU is configured to monitor electrophysiological data, said ECU further configured to map said electrophysiological data onto one of a model of an anatomical structure and an image of said anatomical structure.

21. The system of claim 18, wherein said first medical device comprises a plurality of electrodes mounted on said shaft, and further wherein at least one of said plurality of electrodes comprises one of a pacing electrode, an electrophysiological monitoring electrode, and a positioning electrode.

22. The system of claim 18 further comprising an automated medical device guidance system, said automated medical device coupled with said first medical device and including a controller configured to direct movement of said first medical device.

23. The system of claim 18, wherein said at least one electrode comprises a magnetic coil

24. The medical device of claim 1, wherein said shaft comprises a plurality of electrodes, and further wherein at least one of said plurality of electrodes comprises one of a pacing electrode, an electrophysiological monitoring electrode, and a positioning electrode.
